(19) United States
(12) Patent Application Publication
(10) Pub. No.: US 2004/0260479 A1
(43) Pub. Date: Dec. 23, 2004

(54) Computer software program for graphically displaying genetic linkage disequilibrium, and the method thereof

(75) Inventors: Hitoshi Fujimiya, Mobara-shi (JP); Eiji Nakamura, Urayasu-shi (JP); Hiroki Adachi, Yokohama-city (JP)

Correspondence Address: OMORI & YAGUCHI USA, LLC, EIGHT PENN CENTER, SUITE 1901, 1628 JOHN F. KENNEDY BOULEVARD, PHILADELPHIA, PA, 19103, US

(73) Assignee: Kabushikigaisha DYNACOM, Mobara-shi (JP)

(21) Appl. No.: 10/761,885

(22) Filed: Jan. 21, 2004

(30) Foreign Application Priority Data
Jan. 21, 2003 (JP) 2003-48216

(51) Int. Cl. G06F 19/00; G01N 33/48; G01N 33/50

(52) U.S. Cl. 702/20

(57) ABSTRACT

This invention relates to a computer software program product, including computer readable memory and a computer software program stored on the memory, for calculating linkage disequilibrium values for individual pairs of gene loci for two or more gene polymorphism data groups and displaying results comparatively on a display monitor. The program includes: a color output command for converting the linkage disequilibrium values for individual pairs of gene loci for a first gene polymorphism data group and those for a second gene polymorphism data group into a first color set and a second color set, each color set comprising colors with differently allocated saturation, brightness and density based on the linkage disequilibrium values, and for outputting the two color sets; and a comparative display command for displaying comparative results for the first and second color sets on a display monitor in such a way that comparison of the disequilibrium values between the first and second gene polymorphism data groups can be made.

### CROSS REFERENCE TO RELATED APPLICATIONS

This application claims priority under 35 U.S.C. 119 based upon Japanese Patent Application Serial No. 2003-48216, filed on Jan. 21, 2003. The entire disclosure of the aforesaid application is incorporated herein by reference.

### BACKGROUND OF THE INVENTION

1. Field of the Invention

This invention relates to a method of graphically and comparatively displaying pairwise linkage disequilibrium values that are calculated respectively for a case group and for a control group in gene polymorphism data analyses.

2. Description of the Related Art

In gene polymorphism studies, linkage strengths among various gene loci are often calculated. “Linkage” means that polymorphism at a certain gene locus and that at a target gene locus are genetically transferred as a pair to the descendants. If sufficiently separated from each other on the chromosome, genes undergo a process of random recombination so that after 5 to 6 generations, a state of equilibrium is achieved. This state is called the Hardy-Weinberg equilibrium. If two gene polymorphism loci are physically close to each other, the shift from the Hardy-Weinberg equilibrium is observed. This shift is called “linkage disequilibrium”.

A 2×2 contingency table is created by use of haplotype frequency information at two loci, and the linkage disequilibrium values are obtained based on the shifts from the haplotype frequencies when they are independent.

If the major alleles at the first gene locus and at the second locus are denoted as 1 and their minor alleles are denoted as 3, the respective haplotype frequencies are expressed as follows.

| First gene locus-Second gene locus | Frequency |
|---|---|
| 1-1 | p11 |
| 1-3 | p13 |
| 3-1 | p31 |
| 3-3 | p33 |

Here, the values of p11, p13, p31, and p33 lie between 0 and 1, and p11+p13+p31+p33=1. The linkage disequilibrium value, D, is expressed as follows:

D=p11p33−p13p31.

D can be either negative or positive. It can be rewritten to be a value between 0 and 1, and is redefined as D′, which is a linkage disequilibrium value as well. If D>0 or D=0, the maximum value for D is expressed as follows:

Dmax=min(p1Δ×pΔ3, p3Δ×pΔ1),

where p1Δ is a major allele frequency at the first locus (p1Δ=p11+p13), pΔ3 is a minor allele at the second locus (pΔ3=p13+p33), and similarly, p3Δ is a minor allele frequency at the first locus (p3Δ=p31+p33) and pΔ1 is a major allele frequency at the second locus (pΔ1=p11+p31). If D<0, the minimum value for D is expressed as follows:

Dmin=max(−p1Δ×pΔ1, −p3Δ×pΔ3).

By use of the above expressions, D′ is defined as:

D′=D/Dmax (if D is positive), or

D′=D/Dmin (if D is negative).

In addition, there is another linkage disequilibrium value, r², which is expressed as follows:

r²=D²/(p1Δ×p3Δ×pΔ1×pΔ3).

Additionally, a method using Akaike's Information Criteria (AIC) is available. See, for example, Akaike's Information Criterion for a Measure of Linkage Disequilibrium by K Shimo-Onoda et al., Journal of Human Genetics, Vol. 47 Issue 12 (2002) pp649-655.

It is possible to find a portion having a disease-specific linkage disequilibrium shift by comparing the linkage disequilibrium values for the case group against those for the control group.

However, in the prior art, the linkage disequilibrium values have been simply shown separately in a table format; thus, finding differences between the case group and the control group has been very difficult. Furthermore, the number of single nucleotide polymorphisms employed in tests range generally from several tens to several thousands or more, posing difficulties in identifying the differences.

### SUMMARY OF THE INVENTION

To solve the aforesaid problems, the objective of the present invention is to provide a method of comparatively displaying the linkage disequilibrium values for individual pairs of gene loci for different gene polymorphism data groups. Another objective of the present invention is to calculate the linkage disequilibrium values efficiently using limited computer resources.

According to a first aspect of the present invention, there is provided a computer software program product, comprising computer readable memory and a computer software program stored on the memory, for calculating linkage disequilibrium values for individual pairs of gene loci for two or more gene polymorphism data groups and displaying results comparatively on a display monitor. This program comprises: a color output command for converting the linkage disequilibrium values for individual pairs of gene loci for a first gene polymorphism data group and those for a second gene polymorphism data group into a first color set and a second color set, each color set comprising colors with differently allocated saturation, brightness and density based on the linkage disequilibrium values, and for outputting the two color sets; and a comparative display command for displaying comparative results for the first and second color sets on a display monitor in such a way that comparison of the disequilibrium values between the first and second gene polymorphism data groups can be made.

It is preferable that the comparative display command produces compounded colors, each compounded color obtained by combining the color associated with each pair of gene loci in the first color set and the color associated with the corresponding pair of gene loci in the second color set, and displays an array of the compounded colors on the display monitor as comparative results for the linkage equilibrium values between the first and second gene polymorphism data groups.

According to this configuration, the linkage disequilibrium values for the gene polymorphism case data group and those for the control data group are arranged in a matrix form. This is shown by use of respective colors (colors having different hues) with respective densities based on their linkage disequilibrium values. According to this configuration, differences in the linkage disequilibrium values between comparative data groups can be graphically identified based on the combination of colors and their densities. Said colors can also be achromatic colors such as grayscale colors.

According to one embodiment of the present invention, this program further includes a linkage disequilibrium value calculation command for calculating the linkage disequilibrium values for individual pairs of gene loci for the first and second gene polymorphism data groups respectively.

It is preferable that this program further includes a command for reducing the number of gene loci to be processed. It is further preferable that this command includes: a procedure for calculating information entropy for one or more gene loci; and a procedure for determining the gene loci to be processed based on the information entropy.

According to the one embodiment of the present invention, the information entropy is given by all combinations of minor and major alleles among gene loci and their frequencies.

According to this configuration, the number of gene loci to be processed for the calculation of linkage disequilibrium values can be effectively reduced without reducing the calculation accuracy. In addition, the values of information entropy can also be used as the linkage disequilibrium values. In this case, a high speed calculation processing can be carried out.

According to a second aspect of the present invention, there is provided a computer software program product, comprising computer readable memory and a computer software program stored on the memory, for calculating linkage disequilibrium values for individual pairs of gene loci for two or more gene polymorphism data groups. The program comprising: a command for reading data of a predetermined gene polymorphism data group from a data storage; a command for calculating information entropy for one or more gene loci for the gene polymorphism data group; a procedure for determining gene loci to be processed based on the information entropy; and a command for calculating the linkage disequilibrium values for individual pairs of the gene loci that were determined to be processed and for outputting them for display. It is preferable that the information entropy is given by all combinations of minor and major alleles among gene loci and their frequencies.

According to a third aspect of the present invention, there is provided a computer implemented method for calculating linkage disequilibrium values for individual pairs of gene loci for two or more gene polymorphism data groups and displaying results comparatively on a display monitor. The method comprises: a color output process of converting the linkage disequilibrium values for individual pairs of gene loci for a first gene polymorphism data group and those for a second gene polymorphism data group into a first color set and a second color set, each color set comprising colors with differently allocated saturation, brightness and density based on the linkage disequilibrium values, and for outputting the two color sets; and a comparative display process of displaying comparative results for the first and second color sets on a display monitor in such a way that comparison of the disequilibrium values between the first and second gene polymorphism data groups can be made.

The other features and effects of the present invention can be easily understood by those of ordinary skill in the art by referring to preferred embodiments and drawings illustrating the present invention.

### BRIEF DESCRIPTION OF THE DRAWINGS

FIG. 1 is an overview of a system configuration illustrating one embodiment of the present invention.

FIG. 2A˜FIG. 2C are tables showing the input data and examples of linkage disequilibrium values for the case and control groups.

FIG. 3 is a diagram illustrating a configuration of the color conversion procedure.

FIG. 4 is a flowchart showing the processes in the embodiment.

FIG. 5 is an example of a screen display showing converted colors corresponding to the linkage disequilibrium values for the case and control groups respectively.

FIG. 6 is an example of a display showing the results of the color combining procedure.

FIG. 7 is an example of a display showing the results of the procedure for obtaining differences in the disequilibrium values between the two groups and converting them to colors.

FIG. 8 is a flowchart illustrating the processes in another embodiment.

FIG. 9 is a flowchart illustrating the processes in yet another embodiment.

### DETAILED DESCRIPTION OF THE PREFERRED EMOBDIMENT

An embodiment of the present invention is described below with reference to the accompanying figures.

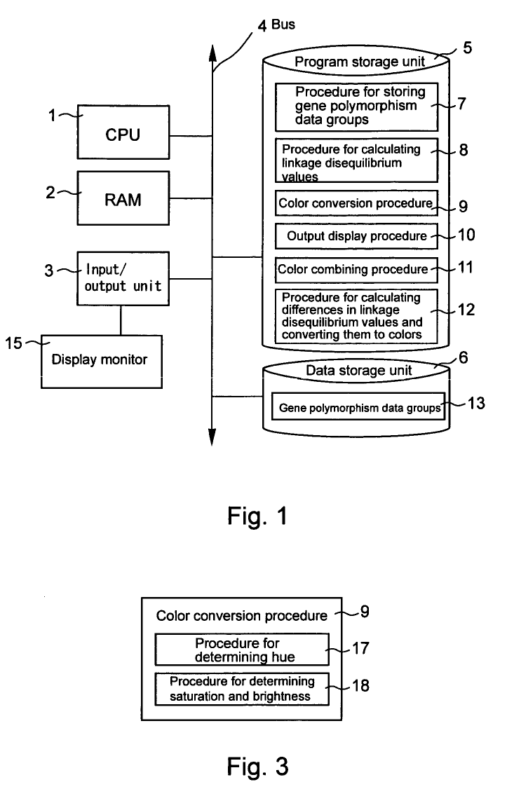

FIG. 1 is an overview of a system configuration with the computer software concerning the embodiment.

This system is comprised of a program storage unit 5 and a data storage unit 6, both connected to a bus 4 to which a CPU 1, a RAM 2 and an I/O unit 3 are also connected. The program storage unit 5 is comprised of the following components: a gene polymorphism data groups storage procedure 7 for storing gene polymorphism data groups 13 in the data storage unit 6; a linkage disequilibrium values calculation procedure 8 for calculating linkage disequilibrium values for pairs of gene loci for each data group by creating a pairwise contingency table; a color conversion procedure 9 for converting the linkage disequilibrium values to a set of colors having color densities based on the values for each data group; a color combining procedure 11 for obtaining combined colors, each combined color obtained by combining the color associated with each pair of gene loci of one data group and the color associated with the corresponding pair of gene loci of another data group; a linkage disequilibrium value differences calculation and color conversion procedure 12 for calculating differences in the linkage disequilibrium values for the corresponding gene loci between the data groups and for converting the differences to a set of colors having colors and densities based on the difference values; and an output display procedure 10 for displaying the color results in a matrix form.

The components 7 through 12 are the commands for the computer system, which is comprised of data and a computer software program installed in the memory medium such as a hard disk via another memory medium (CD-ROM, etc.). These commands 7 through 12 are executed whenever the CPU 1 calls them onto the RAM 2. In addition, a display monitor 15 is connected to the I/O unit 3 to graphically display the outputs obtained from the output display procedure 10.

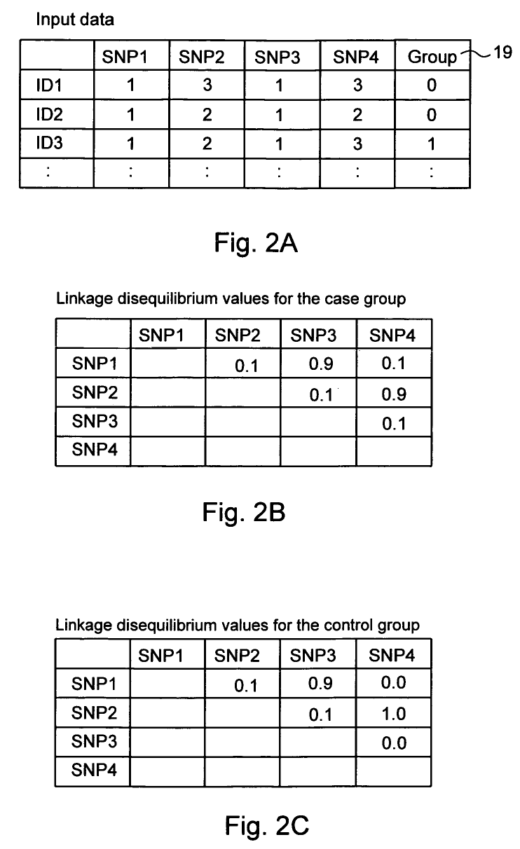

First, the gene polymorphism data groups storage procedure 7 is called and executed on the RAM 2, and the gene polymorphism data groups 13 are stored in the data storage unit 6. FIG. 2A illustrates an example of input data for the case of single nucleotide polymorphisms (denoted as SNP in the figures). This figure shows an example of the test results for human diploid SNPs. In the data, homozygous for the major allele is denoted as “1”, homozygous for the minor allele is denoted as “3”, and heterozygous for both major allele and minor allele is denoted as “2”. The major allele commonly implies the greatest number of polymorphisms. The minor allele implies a small number of polymorphisms. Since these are test results for diploids, cases of two of the same alleles, major allele or minor allele, are called homo and cases with one of each allele are called hetero. In the “group” column 19, “0” represents a case (a disease case) and “1” represents a control (a healthy subject).

Next, the linkage disequilibrium values calculation procedure 8 is executed and the linkage disequilibrium values for various pairs of gene loci are calculated. For this purpose, the gene polymorphism data groups are called from the data storage unit 6 and copied onto the RAM 2. The data are classified into the case group denoted as “0” and the control group denoted as “1”; a 2×2 contingency table for each pair of gene loci is created for each data group. Based on the contingency tables, the linkage disequilibrium values D, D′, r², AIC are calculated.

FIGS. 2B and 2C show examples when the linkage disequilibrium value r² is calculated. FIG. 2B represents a table of the linkage disequilibrium values r² for the case group, and FIG. 2C represents a table of the linkage disequilibrium values r² for the control group. Linkage disequilibrium is not defined for the same gene locus; thus, the diagonal cells are blank. (This situation can be defined as complete linkage.) This is a complete symmetric matrix; thus, only the upper triangular matrix is shown.

For the case of r², if the value is close to 0, this implies that a weak linkage is present between the locations. If the value is close to 1, this implies that a strong linkage is present. Therefore, in the examples shown in FIGS. 2B and 2C, SNP1 and SNP3 are found to have a strong linkage, and SNP2 and SNP4 are also found to have a strong linkage. Therefore, by means of the linkage disequilibrium calculation, differences in the degree of linkages between the two groups can be identified by comparing the linkage disequilibrium values between the case group and the control group. For example, for the case shown in FIGS. 2B and 2C, slight different values are identified in the column for SNP4, indicating that there is a difference in the linkage strength between the case group and the control group.

Subsequently, the color conversion procedure 9 is executed, and specific colors are allocated for respective linkage disequilibrium values. Once color allocations are completed, the output display procedure 10 is executed to replace the disequilibrium values by the allocated colors, which are then displayed in a matrix form on the display monitor 15.

In this embodiment, colors determined by the color conversion procedure are expressed by means of hue (H: 0˜255), saturation (S: 0˜255) and brightness (B: 0˜255) (known as the HSB method). Therefore, the color conversion procedure 9, as shown in FIG. 3, comprises a procedure 17 for determining hue and a procedure 18 for determining saturation and brightness.

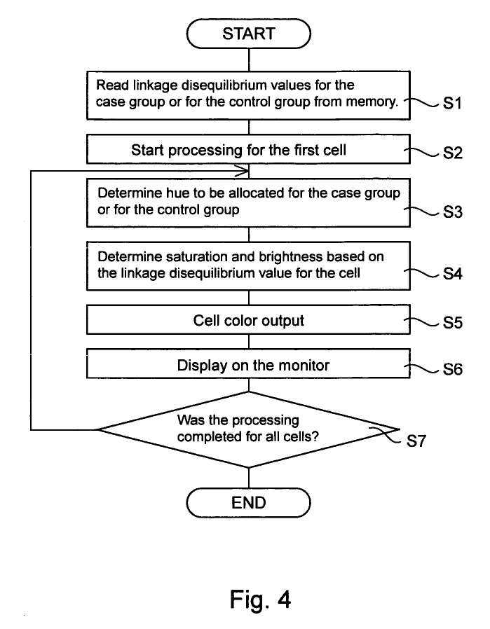

FIG. 4 is a processing flow of the color conversion procedure 9 and the output display procedure 10.

First, the pairwise linkage disequilibrium values for the case group or for the control group are read from the memory (Step S1), and the processing starts successively from the first cell in the matrix (Step S2). Next, the procedure for determining hue 17 determines the hue for the control group or the case group based on a predetermined algorithm. (Step S3) In this algorithm, colors that can be easily combined are selected according to the number of data groups to be compared. In this embodiment, it is programmed that red (0) is allocated for the control group and green (85) is allocated for the case group. (Step S3)

Subsequently, the procedure 18 for determining saturation and brightness allocates saturation and brightness with 256 gradations (values ranging from 0˜256) to the linkage disequilibrium values ranging from 0.0˜1.0. As the linkage disequilibrium value becomes higher, the color is determined to be “darker” with the same hue. According to this scheme, the color in the cell is determined based on the disequilibrium value. (Step S4)

Finally, the output display procedure 10 draws a table on the display monitor 15, and the linkage disequilibrium value in the cell is replaced by the corresponding color (Step S5, Step S6). In this embodiment, the color data originally specified by the HSB are converted to the RGB. Once the above processing is completed for a cell, it is judged if the processing has been completed for all the cells (Step S7). If all the cells are not completed, the aforesaid Steps S3˜S6 are repeated.

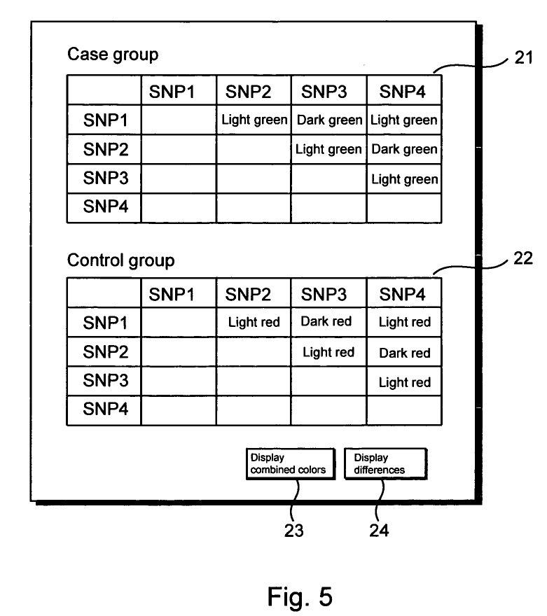

FIG. 5 is a monitor screen illustrating a matrix 21 for the case group and a matrix 22 for the control group. In the actual operation, the actual colors are visually shown, but for convenience, the names of colors are written in FIG. 5. Although the linkage disequilibrium values can be compared visually between the control group and the case group on the screen as shown in FIG. 5, either a menu button 23 “display combined colors” or a menu button 24 “display differences” can be selected on the screen for the purpose of easily identifying the degree of linkage disequilibrium for each cell in this embodiment.

If the button for “display combined colors” is selected, the color combining procedure 11 is executed. In this procedure, the colors expressing the pairwise linkage equilibrium values for the control group and for the case group are combined for each cell by use of the RGB values. The resultant combined colors are displayed in a matrix form on the display monitor 15.

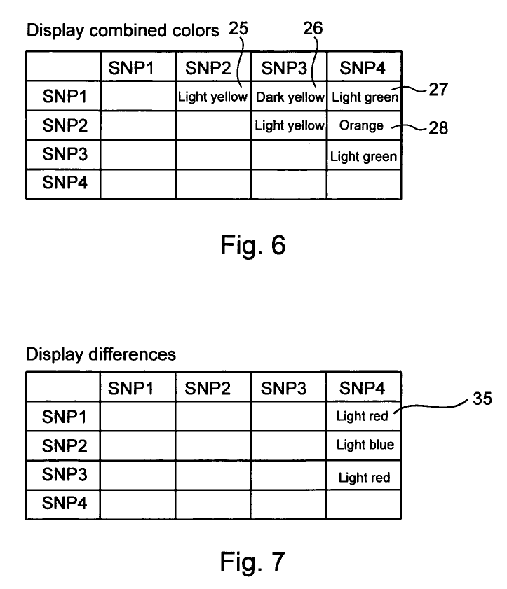

FIG. 6 shows an example of the display of the combined colors. As mentioned above, in the present example, green is allocated for the case group and red is allocated for the control group. Therefore, the results after the color combining are displayed in yellow˜orange˜green, depending on the respective color densities between green and red. For example, the cell 25 in this figure corresponds to the cell with a value of 0.1 for both groups in FIGS. 2B and 2C, and a light green color and a light red color of the same density are combined to present a light yellow color. On the other hand, in the cell 26, the cell value is 0.9 in both groups; they are combined to present a dark yellow color. In the cell 27, the value for the case group is 0.1 and the value for the control group is 0.0; the combined color is a light green color. In the cell 28, the value for the case group is 0.9 and the value for the control group is 1.0; the combined color is a dark yellow color which is close to an orange color due to the slightly stronger red color. These combined colors are obtained by calculating the mean values of two colors to be combined in terms of the R, G, and B values during the color combining procedure 11.

As seen above, when the colors allocated for the case and control groups are combined by direct overlapping, the presence of differences in linkage disequilibrium can be easily identified at a glance based on the resultant color deviation.

Therefore, according to the embodiment of the present invention, there is provided a display method for easily identifying differences in linkage disequilibrium between the case group and the control group.

The aforesaid embodiment is not intended to limit the scope of the present invention. According to the aforesaid embodiment, two groups, a case group and a control group, are compared. However, applications are not limited to this type of case. It is possible to determine the linkage disequilibrium by tabulating other features for displaying the differences. If three or more groups are compared, the differences can be defined with respect to predetermined standards and can be displayed comparatively by allocating hues for respective groups.

Although the differences in the linkage disequilibrium are shown by combining colors in the above example, the differences in the linkage disequilibrium values can be calculated in advance, and then the colors can be allocated to those differences. In this case, the difference in the linkage disequilibrium value for a cell is obtained by subtracting the linkage disequilibrium value of the control group from that of the case group. Blue is allocated for the negative values ranging from −1.0 to 0, and red is allocated for the positive values ranging from 0 to 1.0. Further, the color densities are determined according to the respective absolute values.

FIG. 7 shows an example of displaying the differences. In this figure, the difference in the disequilibrium value for each cell between the case group and the control group is obtained, and only the cells with non-zero value are displayed. The cell 35 represents a case where the value for the case group is greater by 0.1 than that for the control group. If the value for the case group is greater, the color red is allocated. In contrast, blue is allocated if the value in the case group is smaller than that in the control group. That is, blue is allocated for the values ranging from −1.0 to 0 and red is allocated for the values ranging from 0 to 1.0. In both cases, the color becomes darker as the absolute value becomes greater. In this display showing the differences, one can identify at a glance at which locations differences between the two groups are present.

Although colors such as blue and red are used in the embodiment of the present invention, grayscale or other patterns can be used. The present example is made only for the single nucleotide polymorphisms data; however, a pairwise contingency table can be prepared based on data such as micro satellite data. Then, the chi-square values or P values can be calculated and displayed graphically to represent linkage disequilibrium.

As shown in the reference, K Shimo-onoda et al.: Akaike's Information Criterion for a Measure of Linkage Disequilibrium, Journal of Human Genetics, Vol. 47 Issue 12 (2002) pp649-655, it is possible to use the linkage disequilibrium values that are defined as differences between an independent model and a dependent model in the AIC. In the case of using the chi-square values or the linkage disequilibrium values as defined in the AIC, the resultant values range widely from 0 to a great value. The maximum value of the calculated linkage disequilibrium values is obtained first, and various colors are mapped with respect to the maximum value for the graphic display that is visually easy to understand.

Colors can be displayed by means of other display methods. For example, the RGB or the CMYK can be used. After the colors are determined by the HSB system, they may be converted to the RGB system.

In the aforesaid embodiment of the present invention, according to the color combining procedure, two color sets are displayed initially for the control group and the case group respectively as shown in FIG. 5, and subsequently the colors are combined to display the combined colors as shown in FIG. 6. However, the applications are not limited to this mode. The combined colors as shown in FIG. 6 can be displayed directly from the input data without forming the display shown in FIG. 5.

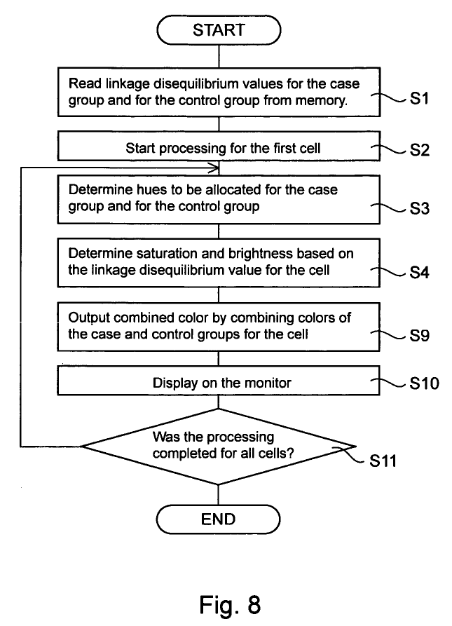

FIG. 8 shows a processing flowchart for the above case. In Step S1 of this figure, the data of the control group and the data of the case group are read. Subsequently, hues (red and green) to be allocated for the control group and for the case group are determined, and the color densities are determined based on the linkage disequilibrium values (Steps S2 through S4).

In the aforesaid embodiment of the present invention, the color data were displayed for the control group and for the case group separately. On the other hand, the combined color for each cell in the present example is determined without such separate displays (Step S9). The resultant combined color for the first cell is displayed on the monitor (Step S11). The above process is repeated for all the cells (Step S11).

In the aforesaid embodiment of the present invention, the linkage disequilibrium values were calculated for all the pairs of gene loci of a gene polymorphisms data group; however, applications are not limited only to this mode. Two or more gene loci may be extracted for the calculation of linkage disequilibrium values. In general, approximately 60% of the analytical results can be obtained by performing an analysis on only 10% of the gene loci in a test. Therefore, a great number of results can be obtained by extracting a small number of gene loci and performing a limited amount of calculations.

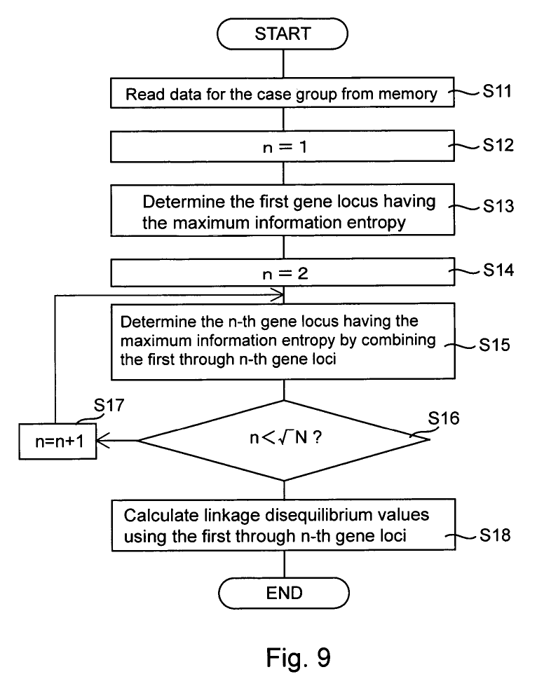

A method of extracting gene loci (a command for extracting gene loci) is explained below with reference to the flowchart shown in FIG. 9. In this method, information entropy is used for the extraction focusing on the minor allele frequencies.

It is preferable to focus on minor allele frequency information, because it is easy to identify genes related to diseases by comparing loci having high minor allele frequencies when they have the same degree of linkage disequilibrium. Also, it is easy to find patients with minor alleles.

In order to extract the gene loci with high minor allele frequencies, gene loci, at which the major allele frequency and the minor allele frequency are antagonistic, are identified first. A method for achieving this is to calculate information entropy for each locus of the case group for comparison. If the major allele frequency and the minor allele frequency are given by p and q (0<p, q<1 and p+q=1) respectively, the information entropy is expressed as follows:

Information entropy=p·log2(1/p)+q·log2(1/q),

where log2( ) is a logarithm with 2 as a base. The information entropy as calculated above clearly represents the degree of antagonism of the allele frequencies at each gene locus. A gene locus having the highest value of the information entropy is initially selected and called a first gene locus (Steps S11˜S13).

Subsequently, a second gene locus is selected in such a way that information entropy becomes the greatest when it is combined with the first gene locus. In order to calculate the information entropy for this case, allele frequencies are tabulated as follows by use of a 2×2 contingency table.

| First gene locus-Second gene locus | Frequencies |
|---|---|
| 1-1 | p11 |
| 1-3 | p13 |
| 3-1 | p31 |
| 3-3 | p33 |

In this case, the information entropy is expressed as follows:

Information entropy=p11·log2(1/p11)+p13_{·log}2(1/p13)+p31·log2(1/p31)+p33·log2(1/p33).

The second gene locus is selected in such a way that the information entropy becomes the greatest when combined with the first gene locus (Steps S14, S15).

The advantage of this technique is that it can be applied to many combinations, not just to pairwise combinations. For the case of combinations of three, frequencies are calculated for all the combinations. For example, if the number of alleles is 2 for the case of single nucleotide polymorphisms, information entropy for the 8 combinations of three loci (p11, p13, p131, p133, p311, p313, p331, and p333) can be calculated as follows:

Information entropy at 3 loci=p111·log2(1/p111)+p113·log2(1/p113)+p131·log2(1/p131)+p133·log2(1/p133)+p311·log2(1/p311)+p313·log2(1/p313)+p331·log2(1/p331)+p333·log2(1/p333).

Using the first and second gene loci which are determined by use of the pairwise method, the information entropy is calculated while combining arbitrary one of the remaining loci as a third gene locus candidate. The one having the greatest information entropy is selected as a third gene locus. Similarly, a fourth candidate and thereafter are obtained in order to select meaningful combinations successively from the plural polymorphisms.

A generalized expression is obtained as follows. Supposing N kinds of patterns are present for the combinations of alleles, these patterns are denoted as A1, A2, A3 . . . , AN. Their pattern frequencies are denoted as pA1, pA2 . . . , pAN. Here, pA1+pA2+ . . . +pAN=1 and 0≦pA1, pA2 . . . , pAN≦1 hold. Using these notations, information entropy H is expressed as follows:

H=pA1·log2(1/pA1)+pA2·log2(1/pA2)+ . . . +pAN·log2(1/pAN).

The extraction for the gene loci will be repeated until the number of extracted gene loci reaches a predetermined number or a predetermined ratio relative to the total number. This number can be predetermined by user, or it can be predetermined by use of a threshold value specified in the system. In this example, if the number of gene loci included in the data group is N, it will be repeated until the number of extracted gene loci reaches √N (Steps S16, S17). Then, the first through n-th gene loci as determined above are outputted to be the data for the calculation of linkage disequilibrium values (Step S18).

When only the group of extracted gene loci is used, the linkage disequilibrium values are not calculated for all combinations. Thus, it is not always possible to achieve an optimal solution, but it is possible to narrow the effective gene polymorphism loci by means of the simple calculation.

Further, in order to reduce the number of gene loci, it is possible to compare the minor allele frequency at each gene locus between the control group and the case group, and extract the ones with a large difference.

Further, a difference in the information entropy between the case group and the control group and a mean information entropy between the two groups can be calculated, and a figure of merit is obtained by multiplying these values as shown in the following:

Figure of merit=a difference in the information entropy between the case group and the control group×a pairwise mean information entropy.

Further, it is possible to extract gene loci with a large pairwise mean information entropy among the top N number of gene loci having a large difference in the information entropy between the case group and the control group.

Furthermore, it is possible to use the information entropy values as the linkage disequilibrium values for carrying out the processes shown in FIGS. 4 and 8.

It is to be understood that the above-described embodiments are illustrative of only a few of the many possible specific embodiments that can represent applications of the principles of the invention. Numerous and varied other arrangements can be readily devised by those skilled in the art without departing from the spirit and scope of the invention.

What is claimed is:

1. A computer software program product, comprising computer readable memory and a computer software program stored on the memory, for calculating linkage disequilibrium values for individual pairs of gene loci for two or more gene polymorphism data groups and displaying results comparatively on a display monitor, said program comprising:
a color output command for converting the linkage disequilibrium values for individual pairs of gene loci for a first gene polymorphism data group and those for a second gene polymorphism data group into a first color set and a second color set, each color set comprising colors with differently allocated saturation, brightness and density based on the linkage disequilibrium values, and for outputting the two color sets; and
a comparative display command for displaying comparative results for the first and second color sets on a display monitor in such a way that comparison of the disequilibrium values between the first and second gene polymorphism data groups can be made.

2. The computer software program according to claim 1, wherein said comparative display command produces compounded colors, each compounded color obtained by combining the color associated with each pair of gene loci in the first color set and the color associated with the corresponding pair of gene loci in the second color set, and displays an array of the compounded colors on the display monitor as comparative results for the linkage equilibrium values between the first and second gene polymorphism data groups.

3. The computer software program according to claim 1, further comprising a linkage disequilibrium value calculation command for calculating the linkage disequilibrium values for individual pairs of gene loci for the first and second gene polymorphism data groups respectively.

4. The computer software program according to claim 3, further comprising a command for reducing the number of gene loci to be processed.

5. The computer software program according to claim 4, wherein said command for reducing the number of gene loci to be processed comprises:
a procedure for calculating information entropy for one or more gene loci; and
a procedure for determining the gene loci to be processed based on the information entropy.

6. The computer software program according to claim 5, wherein the information entropy is given by all combinations of minor and major alleles among gene loci and their frequencies.

7. The computer software program according to claim 5, wherein the value of the information entropy is used as the linkage disequilibrium value.

8. A computer software program product, comprising computer readable memory and a computer software program stored on the memory, for calculating linkage disequilibrium values for individual pairs of gene loci for two or more gene polymorphism data groups, said program comprising:
a command for reading data of a predetermined gene polymorphism data group from a data storage;
a command for calculating information entropy for one or more gene loci for the gene polymorphism data group;
a procedure for determining gene loci to be processed based on the information entropy; and
a command for calculating the linkage disequilibrium values for individual pairs of the gene loci that were determined to be processed and for outputting them for display.

9. The computer software program according to claim 8, wherein the information entropy is given by all combinations of minor and major alleles among gene loci and their frequencies.

10. A computer implemented method for calculating linkage disequilibrium values for individual pairs of gene loci for two or more gene polymorphism data groups and displaying results comparatively on a display monitor, said method comprising:
a color output process of converting the linkage disequilibrium values for individual pairs of gene loci for a first gene polymorphism data group and those for a second gene polymorphism data group into a first color set and a second color set, each color set comprising colors with differently allocated saturation, brightness and density based on the linkage disequilibrium values, and for outputting the two color sets; and
a comparative display process of displaying comparative results for the first and second color sets on a display monitor in such a way that comparison of the disequilibrium values between the first and second gene polymorphism data groups can be made.

11. The method according to claim 10, wherein said comparative display process is a process of producing compounded colors, each compounded color obtained by combining the color associated with each pair of gene loci in the first color set and the color associated with the corresponding pair of gene loci in the second color set, and of displaying an array of the compounded colors on the display monitor as comparative results for the linkage equilibrium values between the first and second gene polymorphism data groups.

12. The method according to claim 10, further comprising a linkage disequilibrium value calculation process of calculating the linkage disequilibrium values for individual pairs of gene loci for the first and second gene polymorphism data groups respectively.

13. The method according to claim 12, further comprising a process of reducing the number of gene loci to be processed.

14. The method according to claim 13, wherein said process of reducing the number of gene loci to be processed comprises:
a process of calculating information entropy for one or more gene loci; and
a process of determining the gene loci to be processed based on the information entropy.

15. The method according to claim 14, the value of the information entropy is used as the linkage disequilibrium value.

16. A computer software program product, comprising computer readable memory and a computer software program stored on the memory, for calculating linkage disequilibrium values for individual pairs of gene loci for two or more gene polymorphism data groups and displaying results comparatively on a display monitor, said program comprising:
a subtraction value output command for obtaining subtraction values, each subtraction value obtained by subtracting the linkage disequilibrium value for each pair of gene loci of a second gene polymorphism data group from the linkage disequilibrium value for the corresponding pair of gene loci of a first gene polymorphism data group, and for outputting the subtraction values; and
a linkage disequilibrium value comparison display command for producing colors corresponding to the subtraction values and for displaying an array of the colors on the display monitor as the linkage disequilibrium value comparison between the first and second gene polymorphism data groups.

17. A computer implemented method for calculating linkage disequilibrium values for individual pairs of gene loci for two or more gene polymorphism data groups and displaying results comparatively on a display monitor, said method comprising:
a subtraction value output process of obtaining subtraction values, each subtraction value obtained by subtracting the linkage disequilibrium value for each pair of gene loci of a second gene polymorphism data group from the linkage disequilibrium value for the corresponding pair of gene loci of a first gene polymorphism data group, and of outputting the subtraction values; and
a linkage disequilibrium value comparison display process of producing colors corresponding to the subtraction values and of displaying an array of the colors on the display monitor as the linkage disequilibrium value comparison between the first and second gene polymorphism data groups.
